(19) United States
(12) Patent Application Publication
(10) Pub. No.: US 2011/0207811 A1
(43) Pub. Date: Aug. 25, 2011

(54) USE OF CELIVARONE FOR REDUCING OCCURRENCES OF CARDIOVASCULAR HOSPITALIZATION

(75) Inventors: Christophe GAUDIN, Saint Cloud (FR); Davide RADZIK, Paris (FR)

(73) Assignee: SANOFI-AVENTIS, Paris (FR)

(21) Appl. No.: 13/028,751

(22) Filed: Feb. 16, 2011

Related U.S. Application Data
(60) Provisional application No. 61/306,254, filed on Feb. 19, 2010.

(51) Int. Cl. A61K 31/343; A61P 9/00

(52) U.S. Cl. 514/469

(57) ABSTRACT

The invention relates to a method of reducing occurrences of cardiovascular hospitalization of a patient by administering to the patient a pharmaceutically effective dose of the compound celivarone, or a pharmaceutically acceptable salt thereof.

The present invention relates to the use of celivarone or a pharmaceutically acceptable salt thereof for the preparation of a medicament for use in the prevention or reducing occurrences of cardiovascular hospitalization.

Celivarone is an antiarrhythmic benzofuran derivative currently in clinical development for the treatment of ventricular arrhythmia. Celivarone, or 2-butyl-3-[4-[3-(dibutylamino)propyl]benzoyl]-1-benzofuran-5-carboxylic acid isopropyl ester fumarate, and other pharmaceutically acceptable salts thereof are described in the international patent application WO 02/16339.

The applicant describes herein that celivarone is effective for the prevention of cardiovascular hospitalization, in a clinical trial using as a calibrator the drug amiodarone, an antiarrhythmic benzofuran derivative commercialized for more than forty years.

The subject-matter of the invention is the use of a compound chosen from celivarone and pharmaceutically acceptable salts thereof for the preparation of a medicament for use in the prevention of cardiovascular hospitalization.

According to the present invention, “cardiovascular hospitalization” designates the hospitalization of a patient due to a cardiovascular disease or event.

According to the instant invention, celivarone or a pharmaceutically acceptable salt thereof is advantageously administered by the oral route.

In an embodiment of the invention, celivarone or a pharmaceutically acceptable salt thereof is administered at a daily dose comprised between 50 and 300 mg, advantageously between 100 and 300 mg.

In another embodiment, celivarone or a pharmaceutically acceptable salt thereof is taken with a meal.

In another embodiment of the invention, celivarone or a pharmaceutically acceptable salt thereof is useful for the prevention of cardiovascular hospitalization in patients having ventricular fibrillation arrhythmia.

In another embodiment of the invention, celivarone or a pharmaceutically acceptable salt thereof is useful for the prevention of cardiovascular hospitalization in patients having an implantable cardioverter defibrillator.

In another embodiment of the invention, celivarone or a pharmaceutically acceptable salt thereof is useful for the prevention of cardiovascular hospitalization in patients having an implantable cardioverter defibrillator and having a left ventricular ejection fraction of 40% or less.

According to the invention, the compound administered in order to prevent cardiovascular hospitalization is 2-butyl-3-[4-[3-(dibutylamino)propyl]benzoyl]-1-benzofuran-5-carboxylic acid isopropyl ester fumarate.

The invention will be more clearly understood by reference to the following example of the invention, outlining the clinical study ALPHEE sponsored by sanofi-aventis.

The following abbreviations shall be used:

ACC/AHA: American College of Cardiology/American Heart Association

ALT: Alanine Aminotransferase

AST: Aspartate Aminotransferase

ATP: Antitachycardia Pacing

ICD: Implantable Cardioverter Defibrillator

LVEF: Left Ventricular Ejection Fraction

VT: Ventricular Tachycardia

VF: Ventricular Fibrillation

Study Objective

The primary objective of the study is to assess the efficacy of celivarone for the prevention of ICD interventions or death. The study also aims at assessing the usefulness of celivarone for reducing occurrences of cardiovascular hospitalization in treated patients.

Study Design

This is a dose-finding multicenter, multinational, randomized, double blind, placebo controlled, parallel arm study including a positive control calibrator arm. Patients are randomized to one of three doses of celivarone or placebo or to the amiodarone calibrator arm.

To be eligible, patients must have an implantable cardioverter defibrillator (see below for detailed inclusion criteria).

The efficacy of celivarone is based on data regarding appropriate ICD intervention obtained by regular ICD interrogation, performed as described below, as well as on data regarding death of the randomized patients, in particular sudden death, and on data regarding cardiovascular hospitalization occurrences.

The study includes a one week screening period, followed by a treatment period scheduled for a minimum duration of 6 months for the last patient recruited and going from the first day of treatment to the end of treatment visit to be done 10-15 days prior to the scheduled study end date.

Patients

Eligible patients are ICD patients with a LVEF of 40% or less, and one of the following criteria:
at least one ICD therapy for VT or VF in the previous month
or ICD implantation in the previous month for documented VTNF.

Concerning the exclusion criteria, a patient is not included if one or more of the following criteria is present

Patients of either sex aged below 21 years (or the age of legal consent of the country).

Women of childbearing potential without adequate birth control or pregnant or breastfeeding women.

Patients with known ICD lead problem (lead dislodgement).

ICD without the following characteristics;
data logging function with cumulative counting of device intervention (shocks and antitachycardia pacing [ATP]),
electrogram storage capabilities,
ventricular demand pacing.

Recent unstable angina pectoris or myocardial infarction (<4 weeks).

History of torsades de pointes.

Genetic channelopathies including congenital long QT syndrome.

Wolff-Parkinson-White syndrome.

Patients in unstable hemodynamic condition such as acute pulmonary edema within 12 hours prior to start of study medication; cardiogenic shock; treatment with intravenous pressor agents; patients on respirator; congestive heart failure of stage New York Heart Association (NYHA) IV within the last 4 weeks; uncorrected, hemodynamically significant primary obstructive valvular disease; hemodynamically significant obstructive cardiomyopathy; a cardiac operation or revascularization procedure within 4 weeks preceding randomization.

Incessant sustained VTNF (VTNF that recurs promptly despite termination attempts) during the three days preceding randomization.

Patients with inappropriate (not triggered by VT nor VF) shocks during the month preceding randomization.

Clinically relevant hematologic, hepatobiliary (ALT, AST>3 times the upper limit of normal at randomization), gastro-intestinal, renal (serum creatinine>221 μmol/l (2.5 mg/dl) at randomization), pulmonary, endocrinologic or psychiatric disease.

Patients treated with oral amiodarone (more than 20 tablets during the 2 months preceding randomization).

Treatments

The investigational products are celivarone (capsules of 50 or 100 mg) or amiodarone (capsules of 200 mg) and their matching placebo in capsules forms.

They are administered by the oral route, with a meal. Indeed, given a significant food effect (drug bioavailability is greatly reduced if taken fasting) it is essential that the study drug is taken with food, such as at the end of a main meal.

The daily doses are 50, 100 or 300 mg for celivarone and 200 mg for amiodarone (with a loading dose of 600 mg daily for 10 days).

Assessment of Investigation Product

VTNF triggered ICD intervention (comprising ICD shocks and ATPs) and death are recorded in the randomized patients.

Cardiovascular hospitalization is an hospitalization with a cardiovascular cause, as assessed by the investigator. Cardiovascular hospitalization analysis is not adjusted for multiplicity in doses and is performed at 5% level; analysis is based on time to first cardiovascular hospitalization, which is estimated using Kaplan-Meier curves. Standard log-rank test is used to compare each celivarone dose curve to the placebo.

What is claimed is:

1. A method of reducing occurrences of cardiovascular hospitalization of a patient, the method comprising administering to the patient a pharmaceutically effective dose of the compound celivarone, or a pharmaceutically acceptable salt thereof.

2. The method according to claim 1, wherein said compound is administered by the oral route.

3. The method according to claim 1, wherein said compound is administered at a daily dose comprised between 50 and 300 mg.

4. The method according to claim 3, wherein said compound is administered at a daily dose of 100 to 300 mg.

5. The method according to claim 1, wherein said compound is taken with a meal.

6. The method according to claim 1, for reducing occurrences of cardiovascular hospitalization in patients having ventricular fibrillation arrhythmia.

7. The method according to claim 1, for reducing occurrences of cardiovascular hospitalization in patients having an implantable cardioverter defibrillator.

8. The method according to claim 1, for reducing occurrences of cardiovascular hospitalization in patients having an implantable cardioverter defibrillator and having a left ventricular ejection fraction of 40% or less.

9. The method according to claim 1 wherein said compound is 2-butyl-3-[4-[3-(dibutylamino)propyl]benzoyl]-1-benzofuran-5-carboxylic acid isopropyl ester fumarate.
